(19) United States
(12) Patent Application Publication
(10) Pub. No.: US 2015/0050495 A1
(43) Pub. Date: Feb. 19, 2015

(54) ZnO-SiO2 NANOPOWDER FOR THE DEVELOPMENT OF LATENT FINGERPRINTS

(71) Applicant: Muhammad Akhyar Farrukh, Lahore (PK)

(72) Inventor: Muhammad Akhyar Farrukh, Lahore (PK)

(21) Appl. No.: 13/968,025

(22) Filed: Aug. 15, 2013

(51) Int. Cl. A61B 5/117

(52) U.S. Cl. CPC A61B 5/1172
USPC 428/402

(57) ABSTRACT

A nanopower composition consisting of zinc oxide and silicon oxide used to develop latent fingerprints on a non-porous surface with a high degree of clarity. The nanopowder composition contains 40-50 percent zinc and 10-20 percent silicon.

### BACKGROUND OF THE INVENTION

Fingerprints are the key and widely used as evidences in criminal investigations since they are internationally recognized means of human identification. Fingerprints are unique to each person and remain unchanged during an individual's lifetime. When a finger touches a surface, secretions are deposited leaving an impression of the finger's ridge pattern, these impression may be latent, patent or plastic impressions. Invisible impressions named as latent fingerprints required physical or chemical treatments to enhance their visualization.

The powder applied to develop the latent fingerprints is evident from 19^{th} century. Over this period, many fingerprint powder formulations have been in use, with each formula consisting of a colorant for contrast and a resinous material for good adhesion. When the fingerprint powder is sprinkled over an affected area, the powder adheres to the oil, sweat or other materials left in a fingerprint enabling it visible. Regular fingerprint powders like titanium dioxide, manganese dioxide, ferric oxide give very clear images by adhering to the moisture and oily constituents of sweat residue through pressure deficit mechanism.

### BRIEF SUMMARY OF THE INVENTION

Metallic powders like silver, gold and gray powder meshed with aluminum and kaolin also gives excellent results and because of the fact that metallic powders have longer shelf lives than other organic-based powders. Luminescent powders containing lanthanide complexes are advantageous because they can be applied on multi-colored surfaces but they are rarely used because of complex method. Nanometallic powders like silver and gold nanoparticles are used to develop latent fingerprints.

### BRIEF DESCRIPTION OF THE DRAWINGS

FIG. 1 depicts FTIR Spectra of ZnO—SiO₂ nanopowder

FIG. 2 depicts Powder XRD pattern of ZnO—SiO₂ nanopowder

FIG. 3A depicts the SEM image of ZnO—SiO₂ nanopowder.

FIG. 3B depicts the EDX graph of ZnO—SiO₂ nanopowder.

FIG. 4. depicts the TEM image of ZnO—SiO₂ nanopowder. The ZnO nanoparticles (dark spots in the image) are well dispersed in amorphous SiO₂ matrix. The mean particle size calculated through TEM images for ZnO—SiO₂ nanopowder was found 32.9 nm. The image also indicates that ZnO nanoparticles are embedded within the SiO₂ matrix and are not deposited on the surface.

FIG. 5 depicts the mechanism of fingerprint development by deposition of ZnO—SiO₂ nanopowder on fingerprint residue.

FIG. 6A depicts latent fingerprints developed on a glazed shiny wrapper using ZnO—SiO₂ nanopowder.

FIG. 6B depicts latent fingerprints developed on a plastic shiny surface(spray bottle cover) using ZnO—SiO₂ nanopowder.

FIG. 6C depicts latent fingerprints developed on a glass surface (reagent bottle) using ZnO—SiO₂ nanopowder.

FIG. 6D depicts latent fingerprints developed on a metallic can using ZnO—SiO₂ nanopowder.

FIG. 6E depicts latent fingerprints developed on a plastic surface (board marker) using ZnO—SiO₂ nanopowder.

FIG. 6F depicts latent fingerprints developed on a plastic surface using ZnO—SiO₂ nanopowder.

FIG. 6G depicts latent fingerprints developed on a calculator surface(textured) using ZnO—SiO₂ nanopowder.

FIG. 6H depicts latent fingerprints developed on a laptop surface (shiny) using ZnO—SiO₂ nanopowder.

FIG. 6I depicts latent fingerprints developed on a cardboard surface (shiny) using ZnO—SiO₂ nanopowder.

FIG. 7 depicts fingerprint developed on wet glass surface using SPR (ZnO—SiO₂ suspension)

FIG. 8A depicts fingerprints developed on plastic shiny surface (spray bottle cover) using ZnO—SiO₂ powder showing third level detail features of pores from which sweat was secreted and second level ridge detail features.

FIG. 8B depicts ridge ending

FIG. 8C depicts bifurcation.

FIG. 9A depicts a comparison with commercial latent fingerprint powder on a cardboard surface.

FIG. 9B depicts a comparison of ZnO—SiO₂ on cardboard surface.

FIG. 9C depicts commercial latent fingerprint powder on metallic can.

FIG. 9D depicts ZnO—SiO₂ on a metallic can.

### DETAILED DESCRIPTION OF THE INVENTION

Metal oxide nanoparticles can be used as fingerprint powder on non-porous surfaces. The effectiveness of the powder that stick to the ridge pattern of the latent fingerprint depends on the shape and size of nanoparticles (nanopowder), as small sized nanoparticles adhere more easily to the latent print residues. Metal oxide nanoparticles like TiO₂, ZnO, Fe₂O₃ and Eu₂O₃ have been used as fluorescent agents, colorants as well as fingerprint powders. Metal oxide nanoparticles are also useful for a large number of other applications including sensors, environmental remediation and electronic materials.

ZnO is used as a nanpowder because of its large value of band gap and due to its excitation binding energy which is important thus having remarkable optical properties. SiO₂ nanoparticles have also much attraction because of applications in many fields such as pharmacy, protection materials used for biomolecules, drug delivery, catalysis, as well as pigments. These nanoparticles have their own electrical, physical and optical properties and have much variation in size. Small particle reagent (SPR) also called wet powdering method can also be used to visualize the latent fingermarks on wet non-porous surfaces. In this work ZnO—SiO₂ nanopowder was synthesized using simple conventional heating method and used to develop latent fingerprints on various surfaces using Powder and SPR methods.

Zinc acetate dihydrate (Zn(CH₃COO)₂.2H₂O), sodium metasilicate pentahydrate (Na₂SiO₃.5H₂O) and sodium hydroxide (NaOH) were purchased from Merck, All chemicals were used without any further purification. Hot plate from CORNING (PC-420D) and High Temperature Tube furnace (HTRH 70-600/18) were used for maintain temperature of the during the synthesis and calcination, respectively. The images of fingerprints were taken by using Nikon camera (Model D-90) of 12 mega pixels. The samples were characterized using Fourier Transform Infra-Red spectroscopy (FTIR) of MIDAC M2000 which determined the presence of metal oxides, Powder X-Ray diffractometer (XRD) of X′pert PRO, PANalytical which was equipped with a copper anode source producing X-rays having wavelength equal to 1.54A. The morphology and elemental composition was studied by using Scanning Electron Microscope-Energy-dispersive X-ray spectroscopy (SEM-EDX) images of Hitachi S3400 using an accelerating voltage of 15.0 kV. Particle size was determined by transmission electron microscope (TEM) of Phillip CM 12 microscope.

ZnO—SiO₂ nanopowder was synthesized by using a simple method that is conventional heating method. The precursors used for synthesis were zinc acetate dihydrate and sodium metasilicate in addition with sodium hydroxide. A 20 mL solution of 0.4 M zinc acetate dihydrate and sodium metasilicate was prepared and heated at 80° C. on magnetic stirrer hotplate and then 20 mL solution of 0.4 M sodium hydroxide was added drop wise for 10 minutes with vigorous stirring at 1100 rpm. The pH of sample solution observed after adding NaOH was 12. The conditions were maintained for 3 hrs. After that, the product was washed using centrifuge machine and rinsed several times with distilled water to remove any impurities left. The product obtained was kept in oven for drying and then was calcined at 700° C. for 1 hr.

Two methods were used including powder dusting method (physical method) and Small Particle Reagent (wet powdering method) for latent fingerprint development. Powder dusting method was applied on nine different surfaces like Cardboard, Glass, Laptop, Metallic Can, Calculator, Board Marker, glazed shiny Wrapper, simple and shiny Plastic. SPR method was used to develop latent finger prints on glass surface. Comparison of ZnO—SiO₂ nanopowder with the commercial white powder was carried out using Powder dusting method on Cardboard and Metallic Can surfaces.

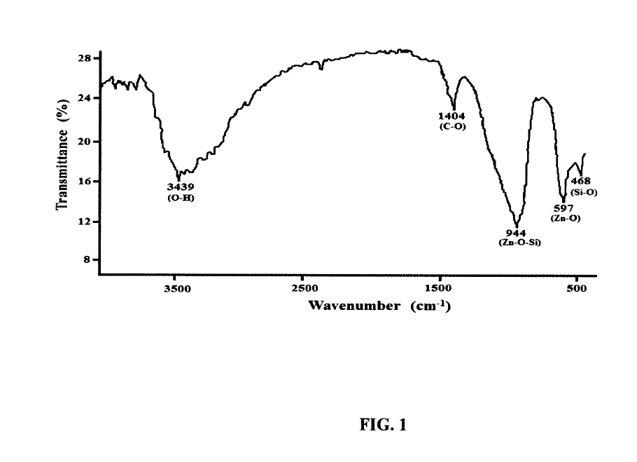

FTIR spectra of ZnO, SiO₂ and ZnO—SiO₂ clearly shows the formation of bond between ZnO and SiO₂. The IR spectrum of ZnO—SiO₂ was acquired in the range of 400 to 4000 cm⁻¹. The Zn—O, Si—O—Si, Zn—O—Si, C—O and O—H show different absorption bands at different wavenumbers as indicated in FIG. 1.

The band at 3439 and 1404 cm⁻¹ is due to stretching vibration of O—H and C—O of water and CO₂ molecules which adsorbed on nanopowder due to air exposure. The strong and sharp band located at 944 cm⁻¹ is due to Zn—O—Si vibration mode. The band located at 597 cm⁻¹ is correlated to zinc oxide. The peak intensity at 468 cm⁻¹ is due to Si—O—Si rocking mode.

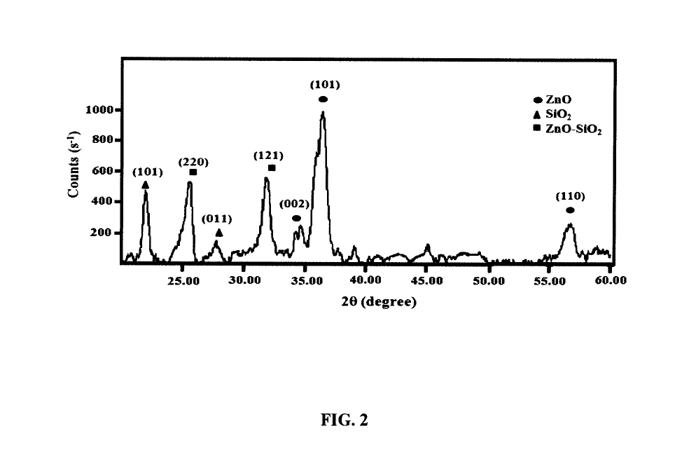

The nature and phase composition of ZnO—SiO₂ nanopowder were identified by X-Ray powder diffractometer with Bragg's angle ranging from 20° to 60°. In FIG. 2 X-Ray diffraction pattern of ZnO, SiO₂ and ZnO—SiO₂ is shown. The presence of sharp peaks of ZnO, SiO₂ and ZnO—SiO₂ indicate of having well crystallite structure.

The presence of ZnO in nanopowder is confirmed by a series of reflection angles (2⊖) at 34.47°, 36.36° and 56.71° having hkl values (002), (101) and (110), respectively with hexagonal plane of ZnO. Similarly, the presence SiO₂ is confirmed by a series of reflection angles (2Θ) at 21.97° and 27.7° having hkl values (101) and (011), respectively with tetragonal plane of SiO₂.

The Spectrum also shows the formation of ZnO—SiO₂ due to the presence of reflection angles (2Θ) at 25.61° and 31.78° having hkl values (220) and (121), respectively with trigonal plane. Thus presences of ZnO, SiO₂ and ZnO—SiO₂ which was indicated by FTIR were also confirmed by using XRD.

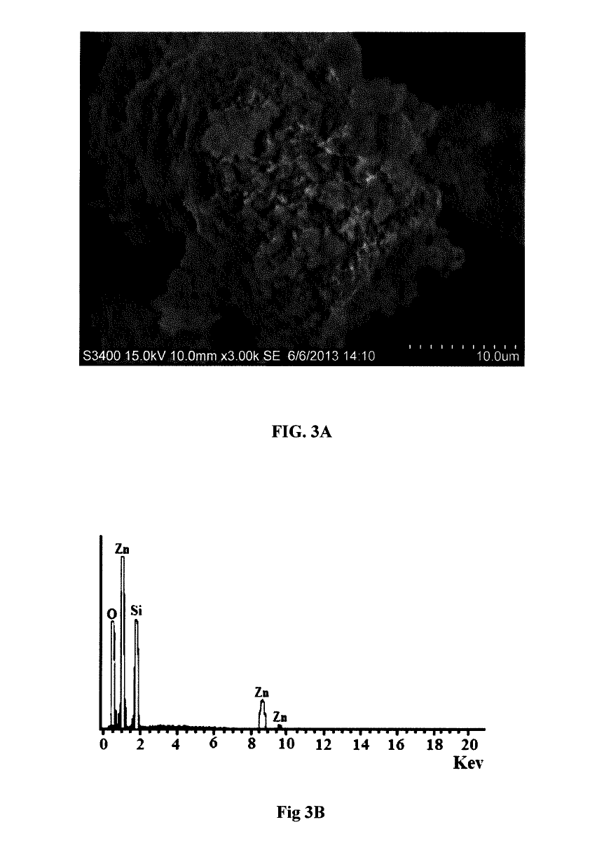

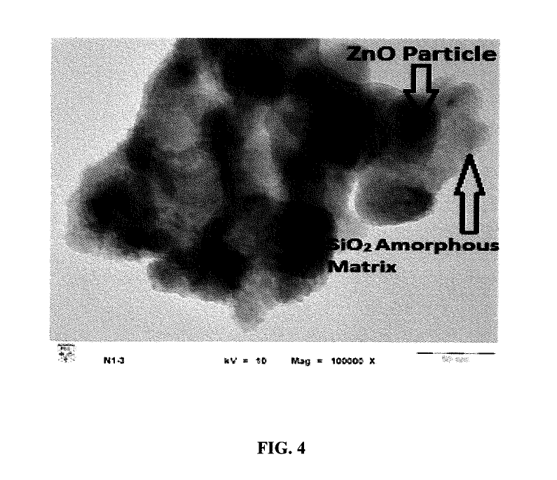

The SEM image as shown in FIG. 3A reveals that morphology of nanopowder is agglomerated. Mostly the agglomerated structures are observed in nanopowder due to the fact that adhesion of nanoparticles to each other occurs due to Van der Waals forces of attraction present among them.

Elemental composition of Zn, Si and P were determined by using EDX analysis. The theoretical percentage was calculated as 46.2% Zn, 19.85% Si and 33.92% O in ZnO—SiO₂ nanopowder. The EDX result showed the actual % of Zn, Si and O is 49.95%, 13.94% and 36.10%, respectively as shown in FIG. 3B.

ZnO—SiO₂ nanopowder was applied to fingerprints on non-porous surface. The nanopowder was applied using powder and SPR methods to develop latent fingerprints.

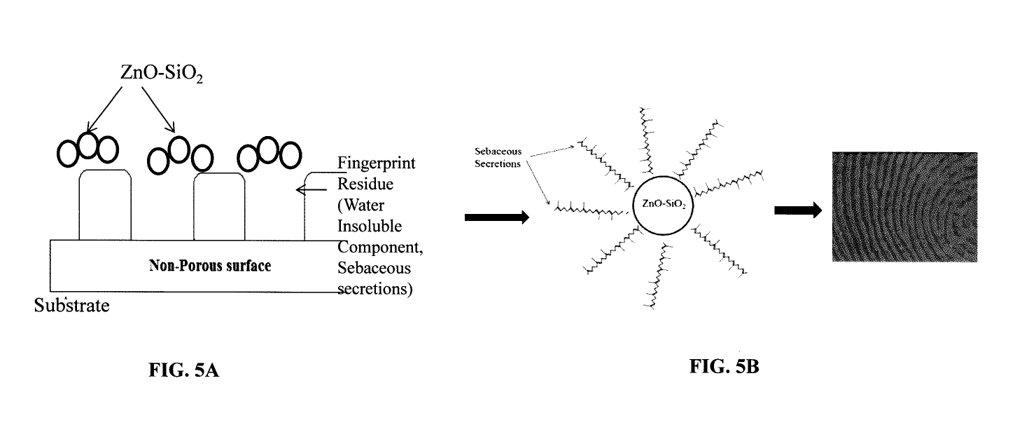

Fingerprint contains many constituents because of the sebaceous and eccrine glands secretions. Sebaceous secretions are present in fingerprints when they were impressed on surfaces after rubbing thumb or fingers over the nose and forehead. ZnO—SiO₂ was applied on these latent fingerprints using powder and brush technique on non-porous surface as shown in FIG. 5. Fingerprints constituents are rich of sebaceous secretions comprising of organic compounds and visibility of latent fingerprint is because of the hydrophobic interaction of ZnO—SiO₂ nanopowder with the fingerprints constituents as shown in FIG. 5.

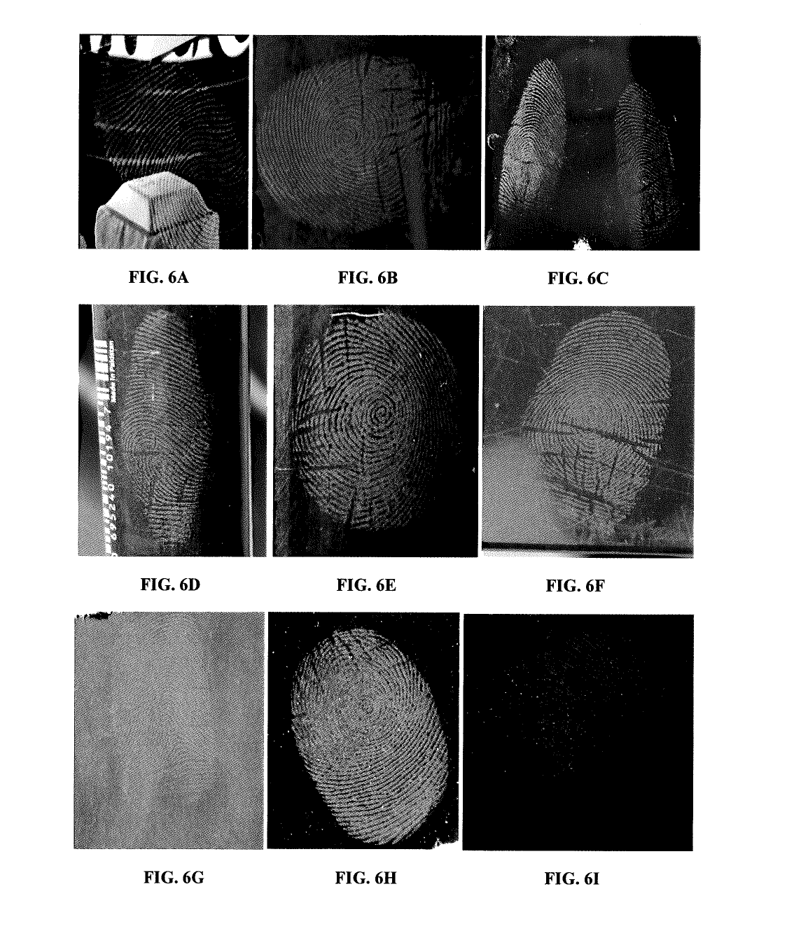

Fingerprints on non-porous surfaces such as cardboard (covering), glass, laptop, Metallic Can, Calculator, Board Marker, glazed shiny wrapper, plastic simple and shiny surfaces were observed by applying ZnO—SiO₂ nanopowder using powder and brush technique as shown in FIG. 6.

ZnO—SiO₂ nanopowder were deposited on all tested surface using powder and brush technique used for developing latent fingerprint on fresh fingerprints.

Fingerprints can be visualized by observing contrast in colour between the background surface and the white powder. The latent fingerprints developed with ZnO—SiO₂ showed excellent ridge details with minimal background staining and better results.

The images above show that powdering method yielded good results on all surfaces except calculator surface due to the fact that it was not smooth although it is non-porous. The plastic surfaces also showed good result because of the existence of electrostatic effect with dry powder on plastic. More intense images can be observed on glazed shiny surface and glass surface. Dark coloured surfaces showed excellent results because of the significant contrast between white powder and background dark colour such as in case of metallic can, glazed shiny surface and laptop surface.

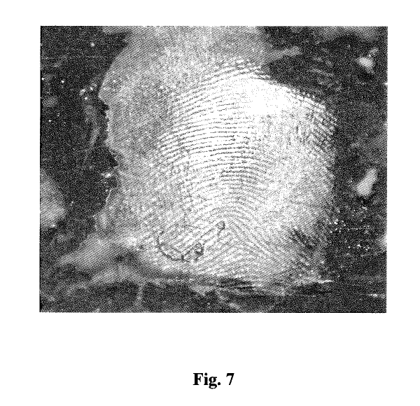

SPR (small particle reagent) technique was used without using any surfactant. FIG. 7 shows the image developed on glass surface using SPR suspension. It revealed the excellent result within few seconds after application of suspension on wet glass surface. SPR yielded well developed fingerprints on glass surface with excellent ridge details.

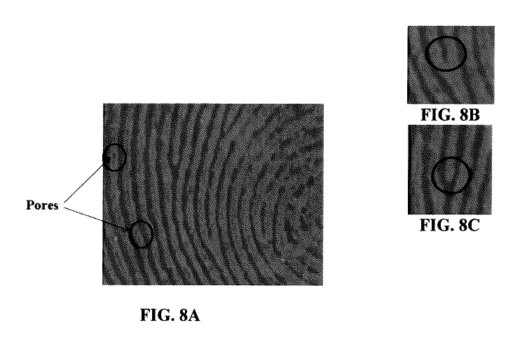

Second and Third level ridge details are the indication of minutiaes (bifurcation, ridge ending etc.) and pores. The development fingerprint using ZnO—SiO₂ nanopowder is so clear that in high resolution second and third ridge details is clearly visible a shown in FIG. 8. This detail is important for identification of fingerprint and presenting evidence in court.

Regular white latent fingerprint powder which is commercially used was purchased from Sirchie Finger Print Laboratories. It is composed of TiO₂ and Zinc steareate as per mentioned in MSDS.

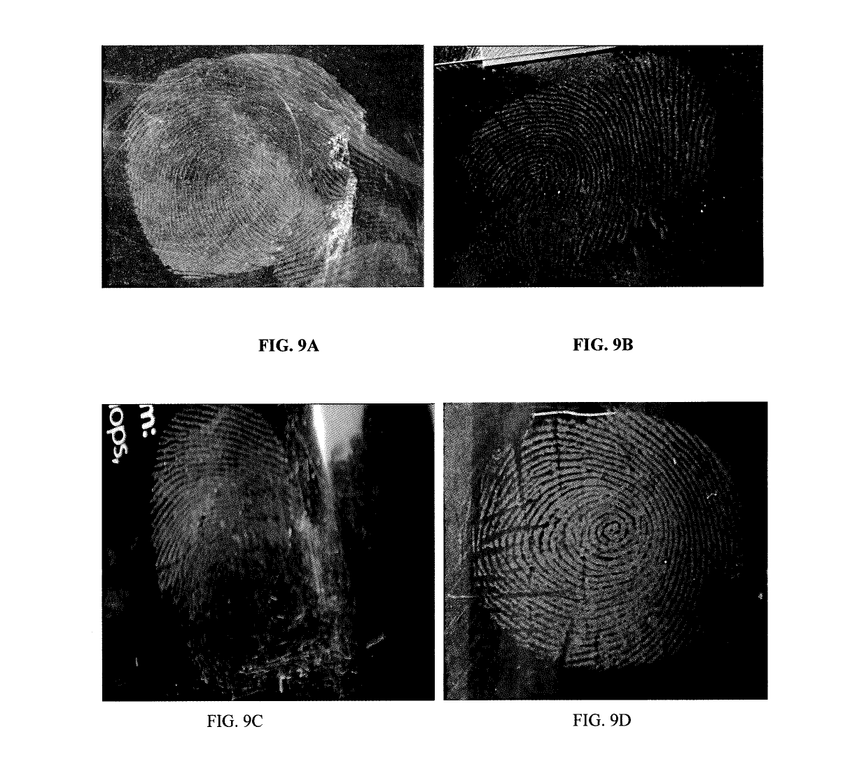

Comparison was done on two surfaces cardboard and Metallic Can as shown in FIG. 9. It was observed that ZnO—SiO₂ applied to latent fingerprints showed distinct ridge pattern other than commercial white powder which stuck to the valley area also diminishing the ridge details of fingerprints. The commercial white powder stuck to the whole surface area where it is applied not specifically on ridges but ZnO—SiO₂ nanopowder only stuck to the ridge areas thus enhancing the visibility of ridge details and increasing its importance as an evidence.

The ZnO—SiO₂ nanopowders can produce clear and sharp images of latent fingerprints on non-porous surfaces with minimal background staining revealing excellent ridge details which is useful in crime investigation. First, second and third level ridge details can also be observed efficiently which are helpful in detailed study of minutae. Other advantages of ZnO—SiO₂ nanopowders include simple synthesis using cheap precursors as well as low toxicity of powder. It is also significant to use it as dry powder as well as a SPR suspension on wet non-porous surfaces. However, ZnO—SiO₂ showed better results on dark colored surfaces than the others examined. ZnO—SiO₂ observed to be a better latent fingerprinting development powder as compared to the commercially available powders.

What is claimed is:

1. A powder composition used for fingerprinting consisting of a mixture of zinc oxide and silicon oxide wherein the size of particles is less than 50 nm

2. The method of claim 1, wherein the content of zinc is between 40 and 50 percent.

3. The powder composition of claim 1, wherein the content of silicon is between 10 and 20 percent.

4. The power composition of claim 1, wherein the said powder composition is used to develop latent fingerprints.

5. The powder composition of claim 1, wherein the said powder composition is used to develop fingerprints on a non-porous surface.
